(19) United States
(12) Patent Application Publication
(10) Pub. No.: US 2017/0241830 A1
(43) Pub. Date: Aug. 24, 2017

(54) Manufacturing Method for Enhanced Acoustic Sensing System

(71) Applicant: Halliburton Energy Services, Inc, Houston, TX (US)

(72) Inventor: Mikko Jaaskelainen, Katy, TX (US)

(21) Appl. No.: 15/506,073

(22) PCT Filed: Oct. 10, 2014
(86) PCT No.: PCT/US2014/060108

§ 371 Date: Feb. 23, 2017

(51) Int. Cl. G01H 9/00; B23K 26/342; A61B 7/02; B33Y 80/00; B23K 26/082; B33Y 10/00; B33Y 50/02; G01N 29/24; B22F 3/105

(52) U.S. Cl. CPC G01H 9/004; G01N 29/2418; B23K 26/342; A61B 7/02; B22F 3/1055; B23K 26/082; B33Y 10/00; B33Y 50/02; B33Y 80/00; B23K 2203/05

(57) ABSTRACT

A method for making a crescent shaped metallic device for attaching to the exterior of the pipe to provide intimate contact for an enhanced acoustic sensing system using a fiber optic sensing cable that can be interrogated by distributed acoustic sensing (DAS) systems wherein the crescent shaped metallic device is used for attaching to the exterior of the pipe and the method of making includes the use of additive layer-wise manufacturing.

### BACKGROUND

This disclosure relates generally to acoustic sensing, and more particularly, to acoustic sensing systems for various types of piping which might include tubing, casing, flow lines, pipe lines etc., in such systems where the signals are concentrated and optimally coupled to a fiber optic sensing cable that can be interrogated using e.g. Distributed Acoustic Sensing (DAS) systems. In particular, this disclosure relates to manufacturing methods for producing the needed uniquely shaped devices of an enhanced acoustic sensing system.

Fiber optic sensing cables are deployed on pipes (tubing, casing, flow lines, pipe lines etc.) today, and the optical fibers are connected to interrogation units like e.g. coherent Rayleigh based Distributed Acoustic Sensing (DAS) systems and/or Distributed Temperature Sensing (DTS) systems. Acoustic energy is transmitted to the cable, and optical fibers, and this acoustic energy can be used to determine e.g. flow rates inside the pipes. The fiber optic cables are commonly strapped outside the pipe.

One of the challenges with the systems currently in use is the coupling from the pipe to the cable housing the fibers. The sensing cables are normally in contact with the pipe, but the contact area is very small, and the sensitivity of the system suffers, which in turn may make the measurements noisy and in some cases not possible.

There is a need then for a technique or method to enhance the sensitivity and performance of these systems.

### BRIEF DESCRIPTION OF THE DRAWINGS

FIG. 1 illustrates a commonly used approach of attaching a sensing cable to a pipe.

FIG. 2 illustrates a device with enhanced acoustic coupling between pipe and sensing cable.

FIG. 3 illustrates the analogy of the use of a stethoscope to collect acoustic energy.

FIG. 4 illustrates a device with enhanced acoustic coupling between pipe and sensing cable using an internal cavity and membrane.

FIG. 5 illustrates the device of FIG. 4 as it is produced and before it is forced down into intimate contact with the pipe.

FIG. 6 illustrates a device as shown in FIGS. 4 and 5 and making use of enhanced acoustic coupling combined with an acoustic filter.

FIG. 7 illustrates a device as shown in FIG. 4 in which one or more internal cavities can be used to aid in directing acoustic energy towards the sensing cable.

### DETAILED DESCRIPTION

In the following detailed description, reference is made to accompanying drawings that illustrate embodiments of the present invention. These embodiments are described in sufficient detail to enable a person of ordinary skill in the art to practice the invention without undue experimentation. It should be understood, however, that the embodiments and examples described herein are given by way of illustration only, and not by way of limitation. Various substitutions, modifications, additions, and rearrangements may be made without departing from the spirit of the present invention. Therefore, the description that follows is not to be taken in a limited sense, and the scope of the present invention will be defined only by the final claims.

Optical fibers are often deployed within fiber optic sensing cables which are deployed on pipes (tubing, casing, flow lines, pipe lines etc.) today, and the optical fibers are connected to interrogation units like e.g. coherent Rayleigh based Distributed Acoustic Sensing (DAS) systems and/or Distributed Temperature Sensing (DTS) systems. Acoustic energy is transmitted to the cable, and optical fibers, and this acoustic energy can be used to determine e.g. flow rates inside the pipes. The fiber optic cables are commonly deployed by being strapped outside the pipe.

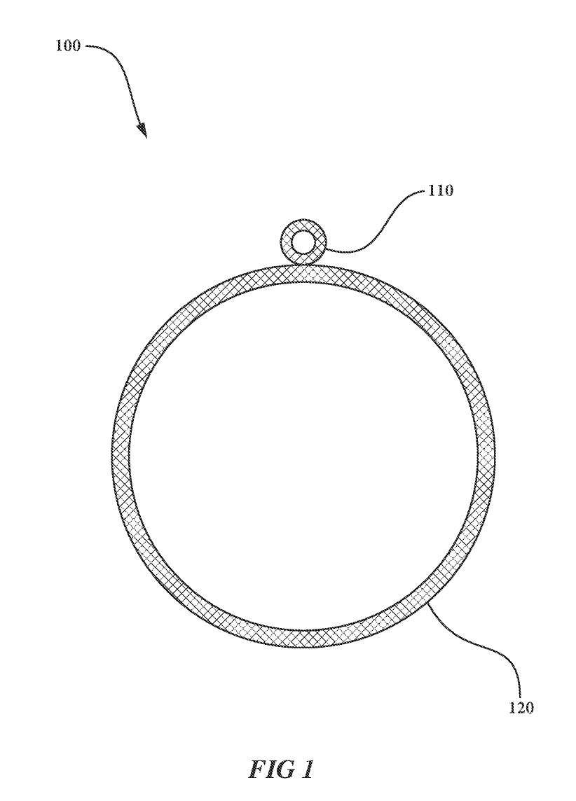

FIG. 1, shown generally as the numeral 100, illustrates a commonly used contact principle between a pipe 120, and an acoustic cable 110 in contact with the pipe. One of the challenges with such systems is the coupling from the pipe to the cable housing the fibers. The sensing cables are normally in contact with the pipe, but the contact area is very small, and the sensitivity of the system suffers, which in turn may make the measurements noisy and in some cases not possible.

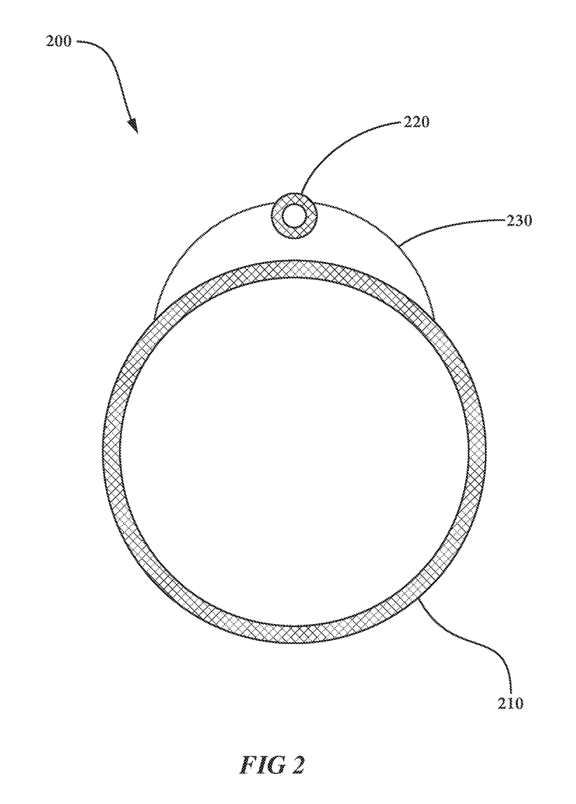

One approach to changing this reality is an enhanced system as shown in FIG. 2, illustrated by the numeral 200. This enhanced system is shown as one embodiment in FIG. 2, in which a fiber optic sensing cable 220 is embedded in a device 230 that is shaped to have a dramatically larger contact area with respect to the pipe, thus improving the path for the acoustic energy to reach the sensing cable. Device 230 is made in a crescent shape that can be tightly clamped or attached along a length of pipe 210 to greatly increase the contact area for picking up acoustic information from the pipe. The fiber optic sensing cable would normally be embedded in the upper part of crescent 230 and the lower part of the crescent would be shaped to be in intimate contact with pipe 210. The crescent shaped device could be applied in a long continuous fashion lengthwise on the pipe or applied along a plurality of sensing positions along the pipe. The application anticipates either of these or combinations.

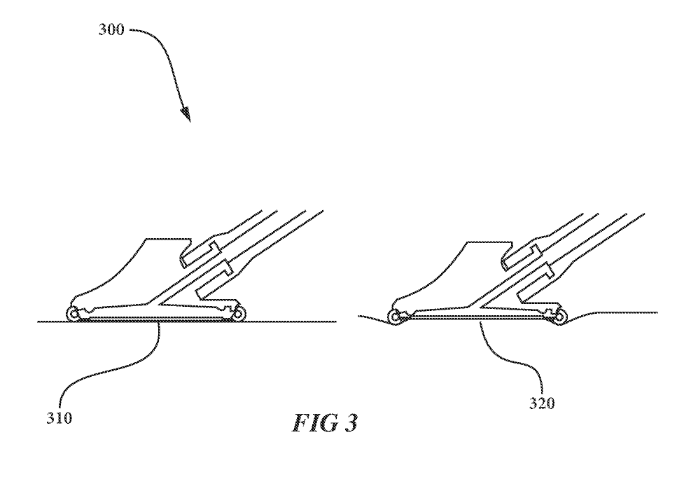

The field of stethoscopes offers an approach for further enhancing the acoustic coupling between a pipe and the sensing cable. Stethoscopes are widely used and are in essence a mechanical amplifier/collector of acoustic energy. For example, FIG. 3, illustrated generally by the numeral 300, illustrates how one type of stethoscope can be used to listen for both low and higher frequency sounds. In example 310 (Bell Mode) a doctor can use light contact with a chest piece and listen for low-frequency sounds, in this bell mode the vibrations of the skin directly produce acoustic pressure waves traveling up to the listener's ears. In example 320 (diaphragm mode) much more pressure is used, pressuring the device down onto the skin, and the device becomes much more sensitive to higher frequency body sounds. In both modes the air cavity acts to gather the acoustic energy and transmit it up the air tubes into the doctors ears.

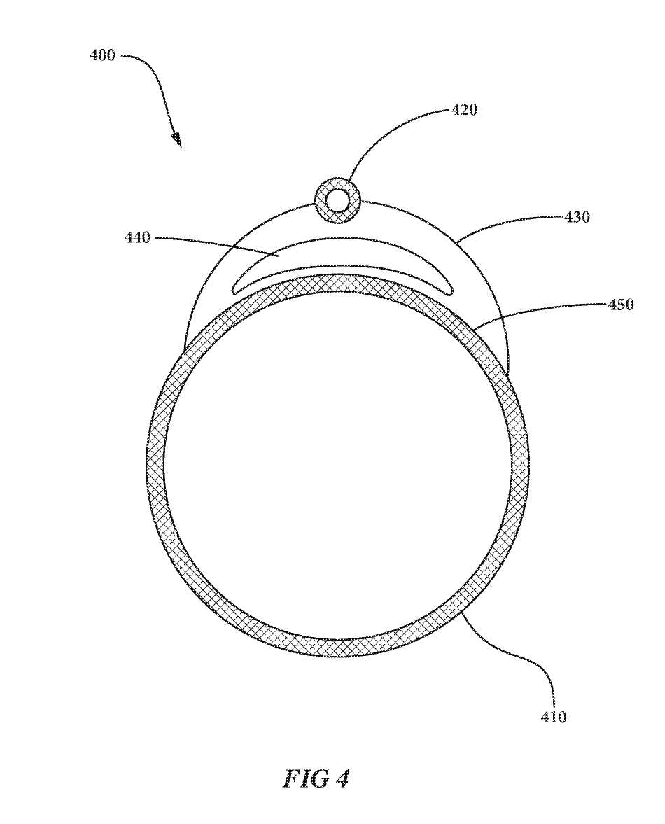

FIG. 4, shown generally by the numeral 400, illustrates another proposed crescent shaped device 430 with enhanced acoustic coupling between pipe 410 and sensing cable 420 that now includes an internal cavity 440 and a membrane portion 450 that is in intimate contact along the length of pipe 410. Device 430 is a crescent shaped piece that is again shaped to have intimate contact with a length of pipe 410 along membrane 450. The fiber optic sensing cable 420 is embedded into device 430. In a manner similar to the stethoscope described earlier the extended internal cavity stretched over an extended piece of the membrane in intimate contact along a length of pipe helps to gather acoustic energy that is transmitted into device 430 and thus into fiber optic sensing cable 420. Alternate combinations of cavity size and membrane thickness can be optimized for different desired frequencies. In addition there can be one or more cavities or channels on either side (not shown). These can provide channels with different acoustic impedance (e.g.air) directing energy towards the sensing cable.

The device will be shaped to couple closely with the pipe and the fiber optic sensing cable, and a compound with suitable acoustic properties can be used at the interfaces between the membrane and pipe and between the fiber optic sensing cable 420 and device 430 to ensure good coupling.

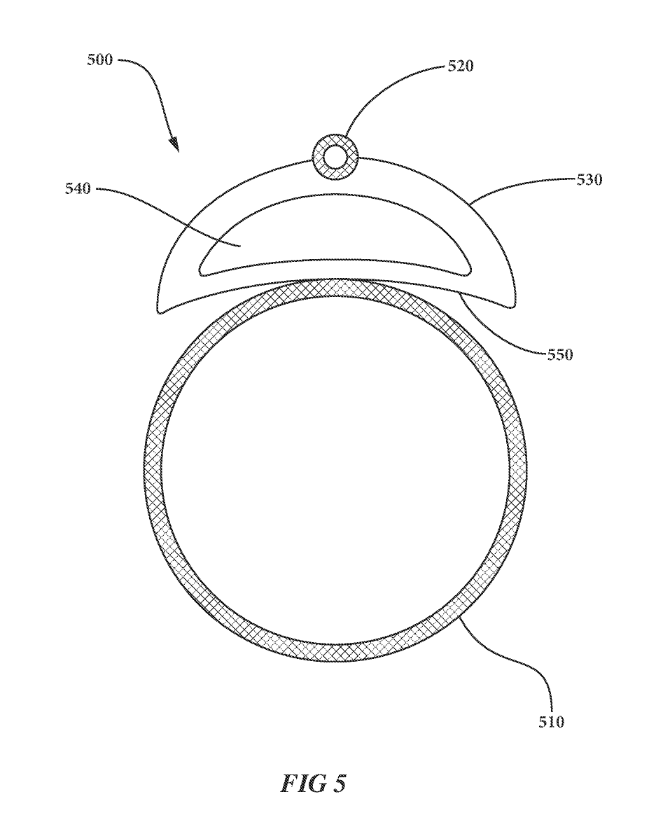

FIG. 5 is illustrating how the crescent shaped metallic device might be created before being applied to a pipe. The device, shown generally by the numeral 500, again illustrates a crescent shaped device 530 and a pipe 510 and sensing cable 520 with an internal cavity 540 and a membrane portion 550 that will be in intimate contact along the length of pipe 510 along membrane 550. The fiber optic sensing cable 520 is embedded into device 530. The design needs to be such that the inner radius 550 is slightly larger than the pipe and would then be clamped down to be in intimate contact with the pipe. The actual curvature of the inner radius 550 would in practice be a very close match to the pipe geometry but is exaggerated as more flat here for illustration purposes only.

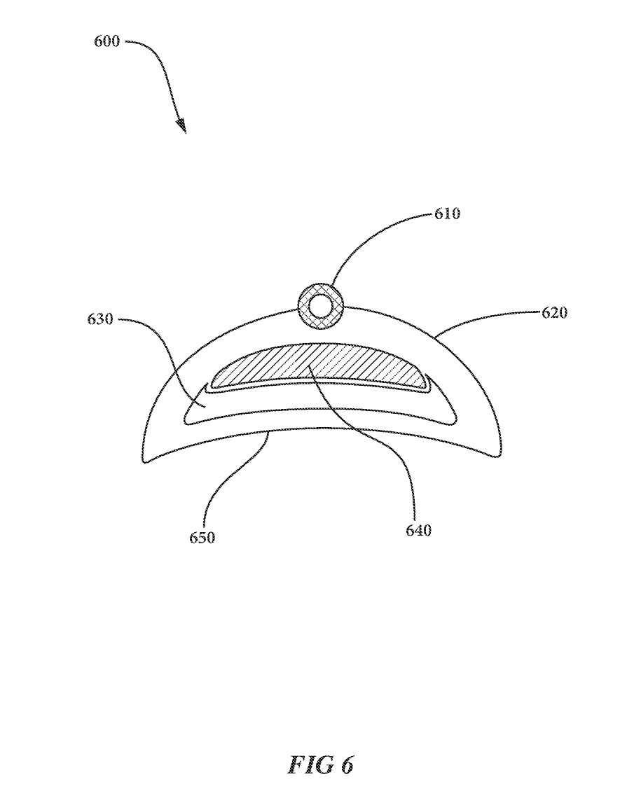

The device of FIG. 4 (or FIG. 5) can be further enhanced by the embodiment shown in FIG. 6, shown generally by the numeral 600. In this embodiment a fiber optic sensing cable 610 is again embedded into a crescent shaped device 620 and again includes an internal cavity 630, but also includes an acoustic filter 640 to block chosen noise bands based on the application. It is known that fluids in general have lower frequency content than gases, and sand/frac proppants/solids may have yet another frequency characteristics. The design of FIG. 6 can be used to combine the type of acoustic filter with the cavity size to provide good acoustic sensitivity for desired frequencies and to screen out the known undesired frequencies. In some embodiments the acoustic filter may completely fill the entire cavity.

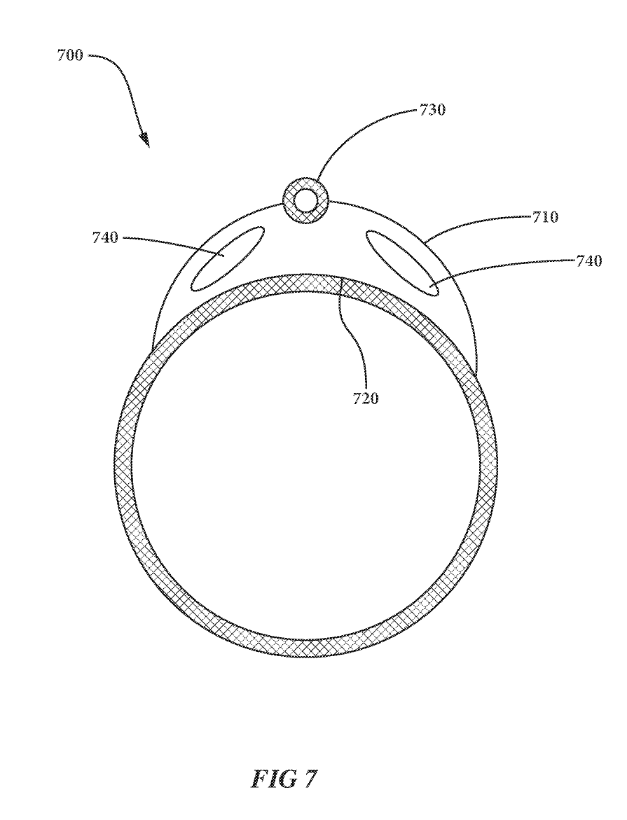

And in FIG. 7, shown generally by the numeral 700 a related but different embodiment of this concept is shown that can introduce a low impedance path between the pipe and the sensing cable where the acoustic energy may travel towards the sensing cable 730. The crescent shaped metallic device 710 may have internal cavities or channels 740 with different acoustic impedance (e.g. air) directing acoustic energy towards the sensing cable 730. FIG. 7 shows 2 internal cavities or channels but more cavities or channels can be used as this may allow different mechanical thickness of walls when compared with a simple air filled cavity as additional walls enhance the structural integrity.

This disclosure assumes any number of suitable materials of construction for the devices shown and described in FIGS. 2, 4, 5, 6, and 7. Some desired options could be Inconel 718, Inconel 625, Titanium TI64, Cobalt Chrome, Stainless Steel 17-4 PH, Alloy 825, or Kovar nickel-cobalt ferrous alloy.

The particular devices described herein create unique manufacturing challenges. The crescent shaped devices of FIGS. 2, 4, 5, 6, and 7, which are all seen endwise, not only have a peculiar outside geometry; they also have one or more interior cavities that extends through the entire length of the device. And in some embodiments these interior cavities may be partially or completely filled with an acoustic material. This type of geometry is extremely difficult to produce with conventional machine shop technologies, such as laser cutting, CNC punching, CNC horizontal milling, vertical CNC machining, etc. These are subtractive technologies and do not handle interior features easily. These types of geometries are also difficult for casting technologies. There are many shapes and geometries that are simply impossible for these approaches. As a result creating the devices of FIG. 2, 4, 5, 6, or 7 would have to be done in separate pieces and in the final analysis joined together with welding or other metal joining approaches.

The proposal of this disclosure is to overcome all of the manufacturing challenges described by an appropriate use of the advancing field of additive manufacturing. Also sometimes called layer wise manufacturing, or 3 dimensional printing, additive manufacturing has the potential advantage of completely eliminating concerns of complex geometries, including the concerns of creating internal geometries of any complexity.

There are many different additive-manufacturing approaches, including stereolithography, selective laser sintering, fused deposition modeling, and others. This disclosure is focused primarily on the high end machines of additive manufacturing, particularly those that can directly create complete metal parts from metals and metal alloys. And the example discussed will be the additive manufacturing approach of Direct Metal Laser Sintering (DMLS). In principle the comparable technology of Selective Laser Melting (SLM) could also be used in this application, and is anticipated in this disclosure. Both are additive layer-wise manufacturing technologies using laser sources to bind metals together but in SLM the material is fully melted rather than sintered as in DMLS. That difference can result in somewhat different physical properties such as crystal structure and porosity.

The DMLS process described herein was developed and commercialized by the EOS firm in Munich, Germany. DMLS developed as an offshoot of the earlier technology of selective laser sintering (SLS) and is similar in many ways but has some unique characteristics do to the increased complexity of sintering metals and metal alloys at higher temperatures and in more demanding environments.

In DMLS a laser is used as the power source to sinter powdered material (typically metal or metal alloy), one layer at a time by aiming the laser at points in a particular layer of the part defined by a 3D CAD model. The 3D CAD model is used to create a data file that defines the object to be made is and further breaks up the three dimensional model into thin horizontal slices and completely defines the boundaries of each of those slices. That data file is then provided to an additive layer-wise manufacturing machine such as a DMLS and is used to direct the machine in successively depositing thin layers of a metal or metal alloy powder onto a target surface and then scanning the aim of a directed energy beam over each layer to successively sinter each thin layer of the three dimensional part as defined by the data file. As each layer is sintered it is also sintered into the previous layer so that a three dimensional part is eventually created that is composed of a plurality of sintered layers.

In one application of such a DMLS machine a high powered 200 watt Yb-fiber optic laser is used as the power source. Inside the build chamber area, there is a material dispensing platform and a build platform along with a recoater blade used to move new powder over the build platform. Parts are built up additively layer-by-layer, typically using layers 20 micrometers thick. This process allows for highly complex geometries to be created directly from the 3D CAD data, fully automatically, in hours and without any tooling. DMLS is a net-shape process, producing parts with high accuracy and detail resolution, good surface quality and excellent mechanical properties.

Since the components are built layer by layer, it is possible to design internal features and passages that could not be cast or otherwise machined. Complex geometries and assemblies with multiple components can be simplified to fewer parts with a more cost effective assembly. DMLS does not require special tooling like castings, so it is convenient for short production runs. Thus any of the device embodiments shown in FIG. 2, 4, 5, 6, or 7 including the interior cavities and an in-built acoustic filter of any possible shape, can be produced in such an additive layer-wise manufacturing machine as described herein. The designer only need to supply the appropriate data file that describes the associated is geometries, including any associated configuration of an internal acoustic filter.

In use any of the proposed systems could operate by transmitting a light pulse (or light pulses) through the optical fibers within the one or more fiber optic sensing cables; interrogating coherent Rayleigh backscatter signals generated by the transmission of the light pulse(s) and acoustic and/or vibration signals; processing the coherent Rayleigh signals to identify acoustic occurrences along the pipe; and embedding the one or more fiber optic sensing cables in a crescent shaped metallic device for attaching to the exterior of the pipe.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

1. A method for making a custom crescent shaped metallic device for attaching to the exterior of a pipe comprising:
a. creating a data file that defines a three-dimensional shape of at least a portion of the custom crescent shaped metallic device; said data file further defining the boundaries of thin cross sectional regions of the custom crescent shaped metallic device;
b. providing the data file to an additive layer-wise manufacturing machine;
c. depositing a first layer of a metal or metal alloy powder onto a target surface;
d. scanning the aim of a directed energy beam over the target surface;
e. sintering a first portion of the first layer of metallic powder with the directed energy beam; said first portion corresponding to a first thin cross sectional region of the custom crescent shaped metallic device;
f. depositing a second layer of metallic powder over the first previously sintered layer;
g. scanning the aim of a directed energy beam over the first sintered layer;
h. sintering a portion of the second layer of metallic powder with the directed energy beam; said second portion corresponding to a second thin cross sectional region of the custom crescent shaped metallic device; including the sub-step of joining the first and second layers during the sintering of the second layer; and
i. depositing successive portions of metallic powder onto the previous sintered layers and sintering each successive portion to produce successive sintered layers joined to a previous sintered layer resulting in the custom crescent shaped metallic device comprising a plurality of sintered layers.

2. The method for making a custom crescent shaped metallic device for attaching to the exterior of a pipe of claim 1 wherein the custom crescent shaped metallic device has one or more internal cavities between the upper part of the crescent shaped device containing the one or more fiber optic sensing cables and a bottom metallic membrane of the crescent shaped metallic device attached against the pipe.

3. The method for making a custom crescent shaped metallic device for attaching to the exterior of a pipe of claim 2 wherein the one or more internal cavities enclose an acoustic filter.

4. The method for making a custom crescent shaped metallic device for attaching to the exterior of a pipe of claim 1 wherein the metal or metal alloy powders are selected from the group consisting of Inconel 718, Inconel 625, Titanium TI64, Cobalt Chrome, Stainless Steel 17-4 PH, Alloy 825, or Kovar nickel-cobalt ferrous alloy.

5. The method for making a custom crescent shaped metallic device for attaching to the exterior of a pipe of claim 2 wherein the metal or metal alloy powders are selected from the group consisting of Inconel 718, Inconel 625, Titanium TI64, Cobalt Chrome, Stainless Steel 17-4 PH, Alloy 825, or Kovar nickel-cobalt ferrous alloy.

6. The method for making a custom crescent shaped metallic device for attaching to the exterior of a pipe of claim 3 wherein the metal or metal alloy powders are selected from the group consisting of Inconel 718, Inconel 625, Titanium TI64, Cobalt Chrome, Stainless Steel 17-4 PH, Alloy 825, or Kovar nickel-cobalt ferrous alloy.

7. The method for making a custom crescent shaped metallic device for attaching to the exterior of a pipe of claim 1 wherein the additive layer-wise manufacturing machine is a Direct Metal Laser Sintering machine.

8. The method for making a custom crescent shaped metallic device for attaching to the exterior of a pipe of claim 1 wherein the additive layer-wise manufacturing machine is a Selective Laser Melting machine.

9. The method for making a custom crescent shaped metallic device for attaching to the exterior of a pipe of claim 2 wherein the additive layer-wise manufacturing machine is a Direct Metal Laser Sintering machine.

10. The method for making a custom crescent shaped metallic device for attaching to the exterior of a pipe of claim 2 wherein the additive layer-wise manufacturing machine is a Selective Laser Melting machine.

11. The method for making a custom crescent shaped metallic device for attaching to the exterior of a pipe of claim 3 wherein the additive layer-wise manufacturing machine is a Direct Metal Laser Sintering machine.

12. The method for making a custom crescent shaped metallic device for attaching to the exterior of a pipe of claim 3 wherein the additive layer-wise manufacturing machine is a Selective Laser Melting machine.
